(19) United States
(12) Patent Application Publication
(10) Pub. No.: US 2023/0390390 A1
(43) Pub. Date: Dec. 7, 2023

(54) CD4+ TFH-LIKE CELLS AS A THERAPEUTIC TARGET

(71) Applicants: Memorial Sloan Kettering Cancer Center, New York, NY (US); Cornell University, Ithaca, NY (US)

(72) Inventors: Jedd Wolchok, New York, NY (US); Roberta Zappasodi, New York, NY (US); Taha Merghoub, Jersey City, NJ (US)

(21) Appl. No.: 18/033,249

(22) PCT Filed: Oct. 21, 2021
(86) PCT No.: PCT/US2021/055992

§ 371 Date: Apr. 21, 2023

Related U.S. Application Data
(60) Provisional application No. 63/104,056, filed on Oct. 22, 2020.

(51) Int. Cl. A61K 39/395; C07K 16/28; A61K 31/5377; A61P 35/00; A61K 31/496; A61K 31/443; A61K 31/4545; A61K 31/4725

(52) U.S. Cl. CPC A61K 39/39558; C07K 16/2818; A61K 31/5377; A61P 35/00; A61K 31/496; A61K 31/443; A61K 31/4545; A61K 31/4725; A61K 2039/505

(57) ABSTRACT

CD4⁺Foxp3⁻PD-1^{hi} T cells (4PD1^{hi}) that increase in tumor-bearing hosts after immune checkpoint blockade (ICB) constitute an unconventional T-cell inhibitory subset with T_{FH}-like features, which can affect the outcome of cancer immunotherapy. Inhibition of the molecular pathway leading to the development of T_{FH} cells and T_{FH}-like 4PD1^{hi} cells improves response to ICB therapy.

### CROSS-REFERENCE TO RELATED APPLICATIONS

This application claims the benefit of U.S. Provisional Application No. 63/104,056, filed on Oct. 22, 2020, which is incorporated herein by reference in its entirety.

### STATEMENT OF GOVERNMENT SUPPORT

This invention was made with government support under CA008748 awarded by the National Institutes of Health. The government has certain rights in the invention.

### COPYRIGHT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### INCORPORATION BY REFERENCE

For countries that permit incorporation by reference, all of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### BACKGROUND

Cytotoxic T-lymphocyte-associated protein-4 (CTLA-4) and programmed cell death protein-1 (PD-1) are the best-characterized immune co-inhibitory receptors that have been successfully exploited as therapeutic targets to promote and reinvigorate immune responses against cancer. Both molecules are induced on T cells upon T-cell receptor (TCR) signaling activation, but with different kinetics. CTLA-4 is usually up-regulated during the initial stage of naïve T-cell activation, and competes with CD28 for the same ligands (CD86 and CD80) expressed on antigen presenting cells (APCs), thus limiting excessive T-cell priming (Fife and Bluestone, 2008; Pentcheva-Hoang et al., 2004). CTLA-4 is also constitutively expressed at high levels on regulatory T cells (T_{regs}), and constitutes one of their immunosuppressive mechanisms (Wing et al., 2008). PD-1 is generally induced during the later phases of an immune response, thus controlling previously activated T cells, typically at the effector sites of immune responses. PD-1 is considered the prototype marker of T-cell exhaustion (Fife and Bluestone, 2008; Keir et al., 2008). The CTLA-4 and PD-1 immune checkpoints are particularly deregulated in tumor-bearing hosts, where chronic ineffective immune responses usually predominate and result in T-cell exhaustion and T_{reg} induction (Wing et al., 2008). These observations provided the rationale for developing strategies to inhibit CTLA-4 and PD-1 as new cancer immunotherapy modalities (Dong et al., 2002; Iwai et al., 2002; Leach et al., 1996; Strome et al., 2003).

Blockade of these immune checkpoints with specific antibodies (anti-CTLA-4 and/or anti-PD-1) has now become a standard of care for various cancer types (Hellmann et al., 2016; Hodi et al., 2010; Larkin et al., 2015; Lutzky et al., 2014; Robert et al., 2015; Weber et al., 2015). The clinical experience accumulated thus far reveals differing activity profiles of CTLA-4 and PD-1 blockade, which can eventually complement each other, as indicated by results from their use in combination (Larkin et al., 2015; Postow et al., 2015; Wolchok et al., 2013).

Despite these successes, immune checkpoint blockade (ICB) still does not benefit a significant proportion of patients with metastatic cancer, and poses a potentially high risk for developing severe immune-related toxicities, in particular when anti-CTLA-4 and anti-PD-1 are combined (Friedman et al., 2016). This underscores the need to achieve better anti-tumor activity in patients receiving ICB therapy.

### SUMMARY OF THE INVENTION

Some of the main aspects of the present invention are summarized below. Additional aspects are described in the Detailed Description of the Invention, Examples, Drawings, and Claims sections of this disclosure. The description in each section of this disclosure is intended to be read in conjunction with the other sections. Furthermore, the various embodiments described in each section of this disclosure can be combined in various different ways, and all such combinations are intended to fall within the scope of the present invention.

We have shown previously that a specific population of T follicular helper (T_{FH})-like cells (CD4⁺Foxp3⁻PD-1^{hi} cells), designated 4PD1^{hi}, have a negative impact on anti-tumor immunity: (i) intra-tumor 4PD1^{hi} accumulation occurs as a function of tumor progression, and (ii) tumor-associated and peripheral 4PD1^{hi} from mice and humans limit effector T-cell (T_{eff}) functions. In addition, we showed that CTLA-4 blockade consistently promotes increases in 4PD1^{hi} cells, while PD-1 blockade reduces their frequency and immunosuppressive function (Zappasodi et al., 2018). We show herein that depletion of T_{FH} cells and T_{FH}-like 4PD1^{hi} cells improves anti-tumor response to ICB therapy.

Accordingly, the invention provides a method of treating cancer or improving treatment response in a patient undergoing ICB therapy. In certain embodiments, the patient displays an increased frequency of 4PD1^{hi} cells compared to the average frequency of 4PD1^{hi} cells in healthy subjects.

One embodiment provides a method of improving response of a patient to ICB therapy, the method comprising administering to the patient an agent that reduces frequency or function of T_{FH} cells and/or 4PD1^{hi} cells in the patient, wherein administration of the agent is commenced prior to or concurrently with the ICB therapy. Another embodiment provides a pharmaceutical composition comprising an effective amount of an agent that reduces frequency or function of T_{FH} cells and/or 4PD1^{hi} cells in a patient for use in improving response of the patient to ICB therapy.

A particular embodiment provides a method of improving response of a patient to anti-CTLA-4 ICB therapy, the method comprising administering to the patient an effective amount of a B-cell lymphoma 6 (BCL6) inhibitor, wherein administration of the BCL6 inhibitor is commenced prior to or concurrently with administration of a CTLA-4 inhibitor. Another embodiment provides a pharmaceutical composition comprising an effective amount of a BCL6 inhibitor for use in improving response of a patient to anti-CTLA-4 ICB therapy.

Another particular embodiment provides a method of improving response of a patient to anti-CTLA-4 ICB therapy, the method comprising administering to the patient an effective amount of an enhancer of zeste homolog 2 (EZH2) inhibitor, wherein administration of the EZH2 inhibitor is commenced prior to or concurrently with administration of a CTLA-4 inhibitor. A further embodiment provides a pharmaceutical composition comprising an effective amount of an EZH2 inhibitor for use in improving response of a patient to anti-CTLA-4 ICB therapy.

In one aspect, the agent that reduces frequency or function of T_{FH} cells and/or 4PD1^{hi} cells comprises a BCL6 inhibitor. In some embodiments of the invention, the BCL6 inhibitor is 79-6. In another aspect, the agent that reduces frequency or function of T_{FH} cells and/or 4PD1^{hi} cells comprises an EZH2 inhibitor. In certain embodiments, the EZH2 inhibitor is EPZ-6438 (tazemetostat). In other embodiments, the EZH2 inhibitor is a dual EZH2/1 inhibitor.

The agent can be administered to the patient starting, for example, between one and seven days prior to ICB therapy or starting, for instance, on the same day as ICB therapy. In certain embodiments, the agent is administered to the patient starting between one and four days prior to administration of the ICB therapy. In one embodiment, a course of the agent is administered throughout a course of the ICB therapy.

In some embodiments, the ICB therapy comprises a CTLA-4 inhibitor, a PD-1 inhibitor, or a combination thereof. In one embodiment, the ICB therapy is a CTLA-4 inhibitor. In particular embodiments, the CTLA-4 inhibitor is selected from the group consisting of ipilimumab and tremelimumab.

T_{FH} cell frequency and/or 4PD1^{hi} cell frequency can be measured, for example, using a method comprising immunohistochemistry, flow cytometry, and/or gene expression signature. In one embodiment, the flow cytometry is fluorescence-activated cell sorting (FACS).

In certain aspects of the invention, the frequency or function of T_{FH} cells and/or 4PD1^{hi} cells is measured in a peripheral blood sample from the patient. In other aspects of the invention, the frequency or function of T_{FH} cells and/or 4PD1^{hi} cells is measured in a tumor biopsy sample from the patient.

In some embodiments, the patient has cancer. In one embodiment, the cancer is melanoma. In one embodiment, the cancer is non-small cell lung cancer (NSCLC).

### BRIEF DESCRIPTION OF THE FIGURES

FIG. 1 shows that targeting T_{FH} cells improves the activity of CTLA-4 blockade. In the experiments shown in Panels A and B, B16-melanoma-bearing CD4^{cre}Bcl6^{fl/fl} or Bcl6^{fl/fl} control mice (ctrl) (n=3-5/group) were treated with anti-CTLA-4 (clone 9D9, 100 μg) or the isotype control as indicated and monitored for tumor growth (Panel A; mean±SEM, 2-way ANOVA with Bonferroni correction). Frequencies of T_{FH} cells (CXCR5⁺PD-1⁺Foxp3⁻CD4⁺ T cells) and 4PD1^{hi} cells were quantified in spleen and tumor by flow cytometry after treatment (Panel B; mean±SEM, 2-sided unpaired t test). In the experiments shown in Panels C and D, Recombination activating gene (RAG) knockout (KO) mice were reconstituted with mixed CD4 KO and CXCR5 KO or mixed CD4 KO and wild type (WT) bone marrows. One to two months later, lymphoid reconstitution in recipient mice was evaluated by flow cytometry analysis of peripheral blood (PB). Two to three months after bone marrow transplantation (BMT), recipient mice were injected intradermally with B16 cells; seven days later, mice were treated with anti-CTLA-4 or isotype control for 4 administrations, 3 days apart (suboptimal treatment). Mice were monitored for tumor growth (Panel C; mean±SEM, 9-10 mice/group; 2-way ANOVA with Bonferroni correction). Frequencies of T_{FH} cells (Bcl6⁺PD-1⁺Foxp3⁻CD4⁺ T cells) and 4PD1^{hi} cells in spleen and tumor were quantified by flow cytometry one day after completion of treatment (Panel D; mean±SEM, 9-10 mice/group; 2-sided unpaired t test). In the experiments shown in Panels E and F, B16-melanoma-bearing WT or μMT B-cell deficient mice were treated daily with the selective Bcl6 inhibitor 79.6 (Bcl6i) or control vehicle, starting on day 4 after tumor implantation, and with anti-CTLA-4 or the isotype control, as indicated. Mice were monitored for tumor growth (Panel E; left graph, mean±SEM, 9-10 mice/group; right graph, mean SEM of 5 mice/group; 2-way ANOVA with Bonferroni correction). Frequencies of T_{FH} cells (CXCR5⁺PD-1⁺Foxp3⁻CD4⁺ T cells) and 4PD1^{hi} cells in spleen and tumor were quantified by flow cytometry one day after completion of treatment (mean±SEM, 3-5 mice/group, 2-sided unpaired t test). *P<0.05, **P<0.01, ***P<0.001.

FIG. 2 shows loss of immunosuppression of intratumor 4PD1^{hi} in mice with a defective T_{FH} program (harboring CXCR5 KO CD4⁺ T cells). In the experiment shown in Panel A, CD4KO:CXCR5KO and CD4KO:WT bone marrow chimera mice were generated, implanted with B16, and treated with anti-CTLA-4 as in FIG. 1, Panel C. One day after completion of treatment, 4PD1^{hi}, convention T cells (Tconv), and T_{regs} were FACS-sorted from pooled tumors as indicated in the gating strategy (black, stained cells; gray, cells stained with isotype control). Panel B shows representative plots and quantification of proliferating CellTarceViolet (CTV)-labeled CD8⁺ T cells activated with anti-CD3, and co-cultured for 72 hours with 4PD1^{hi}, Tconv, and T_{regs} from tumors of CD4KO:CXCR5KO or CD4KO:WT bone marrow chimera mice treated as in Panel A (n=2-6, mean±SD; 2-sided unpaired t test). Panel C shows quantification of mRNA expression by real time quantitative PCR of the indicated genes in 4PD1^{hi}, Tconv, and T_{regs} isolated from tumors as in Panel A (n=2, mean±SD; 2-sided unpaired t test). *P<0.05, **P<0.01, ***P<0.001.

FIG. 3 shows higher expression of EZH2 in 4PD1^{hi} cells as compared to other CD4⁺ T-cell subtypes. In the experiment shown in Panels A and B, CD4⁺Foxp3⁻PD-1⁻ Tconv, Foxp3⁺PD-1⁻T_{regs}, and 4PD1^{hi} cells were isolated from the spleen of naïve mice and from peripheral blood monocyte cells (PBMC) of human healthy donors. RNA sequencing from these cell subsets was analyzed to determine mRNA expression levels of EZH2 and EZH1 in each of the T-cell subtypes from the mouse spleen (Panel A; mean±SEM, n=3-5, 2-sided unpaired t test) and human PBMC (Panel B; mean±SEM, n=5, 2-sided unpaired t test). In addition, CD4*Foxp3⁻PD-1⁻Tconv, Foxp3* T_{regs}, and 4PD1^{hi} cells were isolated from the spleen of mice who were treated with an anti-CTLA-4 antibody or with an immunoglobulin G (IgG) control. Single-cell RNA-sequencing from these sell subsets was analyzed to determine EZH2 and EZH1 mRNA expression, and the proportion of positive cells expressing EZH2 and EZH1 was calculated (Panel C; data are from n=3 pooled spleens per condition, and n=2792-4914 cells per cell type and condition were analyzed).

### DETAILED DESCRIPTION OF THE INVENTION

Unless defined otherwise, all technical and scientific terms used herein have the same meaning as commonly understood by one of ordinary skill in the art to which this invention is related. For example, The Dictionary of Cell and Molecular Biology (5th ed. J. M. Lackie ed., 2013), the Oxford Dictionary of Biochemistry and Molecular Biology (2d ed. R. Cammack et al. eds., 2008), and The Concise Dictionary of Biomedicine and Molecular Biology (2d ed. P-S. Juo, 2002) can provide one of skill with general definitions of some terms used herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/of” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Système International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10. Where a numeric term is preceded by “about,” the term includes the stated number and values ±10% of the stated number. The headings provided herein are not limitations of the various aspects or embodiments of the invention, which can be had by reference to the specification as a whole. Accordingly, the terms defined immediately below are more fully defined by reference to the specification in its entirety.

Wherever embodiments are described with the language “comprising,” otherwise analogous embodiments described in terms of “consisting of” and/or “consisting essentially of” are included.

BCL6, or B-cell lymphoma 6, is a zinc finger transcription repressor that is overexpressed/mutated in lymphoid malignancies, for example, diffuse large B cell lymphoma. BCL6 plays a role in the development of T_{FH} cells.

T_{FH} cells are a subset of CD4^{m} T cells found in the B cell follicles of secondary lymphoid organs. T_{FH} cells constitutively express CXC chemokine receptor 5 (CXCR5), which enables T cells to migrate to the B cell zone. BCL6 is normally responsible for retaining germinal center T_{FH} cells, as well as B cells, in the follicles (Mlynarczyk et al., 2019).

“4PD1^{hi}” are Foxp3⁻PD-1^{hi} CD4⁺ T_{FH}-like cells that fine tune the immune system by suppressing certain immune functions while potentiating others (Zappasodi et al., 2018). They are a subset of immunosuppressive CD4⁺ T cells with T_{FH}-like features, and express the highest levels of PD-1 within the CD4⁺ T cell pool. The observation that 4PD1^{hi} cells increase and accumulate within the tumor microenvironment as a function of tumor growth indicates that persistent tumor-antigen exposure may facilitate and sustain their generation. For example, the average frequency of 4PD1^{hi} cells as a percentage of CD4⁺ T cells is increased in advanced melanoma patients and in NSCLC patients, compared with that in healthy donors, particularly in the tumor microenvironment, as shown in Table 1.

TABLE 1

| Sample Source | Avg. Freq. |
|---|---|
| Healthy donor PB (n = 7) | 1.96 |
| Melanoma patients PB (n = 47) | 3.09 |
| Melanoma patients TM (n = 10) | 10.32 |
| NSCLC patients PB (n = 51) | 6.01 |
| NSCLC patients TM (n = 10) | 21.53 |

PB: peripheral blood sample;
TM: tumor biopsy

We demonstrate herein that targeting the T_{FH} differentiation pathway can potentiate the activity of ICB therapy. In one embodiment, the ICB therapy is CTLA-4 blockade. In addition, our results indicate that when the T_{FH} lineage is compromised, the 4PD1^{hi} cells that develop or are retained in the tumor after ICB therapy no longer suppress T cells and may acquire Th1-like functions. In particular, we found that the response of a subject to ICB therapy can be improved by co-administering an agent that reduces the frequency and/or function of T_{FH} cells and/or 4PD1^{hi} cells. Improvement is assessed in comparison to a control population.

The term “immune checkpoint blockade,” “ICB,” or “ICB therapy,” as used herein, refers to the administration of one or more inhibitors of one or more immune checkpoint proteins or their ligand(s). Immune checkpoint proteins include, but are not limited to, cytotoxic T lymphocyte-associated antigen 4 (CTLA-4), also known as CD152, programmed cell death protein 1 (PD-1), also known as CD279, lymphocyte-activation gene 3 (LAG-3), also known as CD223, and T cell immunoglobulin mucin (TIM-3), also known as HAVcr2.

EZH2, or enhancer of zeste homolog 2, is the enzymatic component of the polycomb repressive complex 2 (PRC2) and catalyzes histone 3 lysine 27 trimethylation (H3K27me3) at gene promoters, thus repressing transcription. EZH2 cooperates with BCL6 in B cells to execute a gene repressor program that orchestrates the germinal center reactions (Béguelin et al., 2016). The epigenetic activity of EZH2 has also been shown to contribute to the phenotypic stability and immunosuppression function of T_{regs} (duPage et al., 2015).

EZH1, or enhancer of zeste homoog 1, is the EZH2-paralog.

By “subject” or “individual” or “patient” is meant any subject, preferably a mammalian subject, for whom diagnosis, prognosis, or therapy is desired. Mammalian subjects include humans, domestic animals, farm animals, sports animals, and zoo animals including, e.g., humans, non-human primates, dogs, cats, guinea pigs, rabbits, rats, mice, horses, cattle, and so on.

A “control” patient or population is one that has not been subjected to methods of the invention. Control patients, or subjects in a control population, have the same disease or disorder as the patient being compared to the control population. For example, a clinical outcome of a cancer patient who is subjected to a method of the invention, e.g., treatment with ICB therapy and an agent that reduces T_{FH} cell and/or 4PD1^{hi} cell frequency and/or function, is compared with the average (median) outcome of subjects having the same type of cancer who were not subjected to methods of the invention, e.g., who received the same ICB therapy but did not receive an agent that reduces T_{FH} cell and/or 4PD1^{hi} cell frequency and/or function.

In one embodiment, a patient subjected to a method of the invention has an improved response if the patient's survival is longer than the median survival of patients having the same type of disorder as the patient, who were not subjected to a method of the invention. Survival can be overall survival, i.e., length of time a patient lives, or progression-free survival, i.e., length of time a patient is treated without progression of the disease. Survival can be measured from the date of diagnosis or from the date that treatment commences. Overall survival, median overall survival, progression-free survival, and median progression-free survival can be determined by methods known in the art and/or by those described herein. Improvement is preferably statistically significant as analyzed, for example, by Wilcoxon matched-pairs signed rank test, log-rank (Mantel-Cox) test, or paired t-test.

In one embodiment, improved response can be measured by known methods appropriate to the disease type, for instance, using Response Evaluation Criteria in Solid Tumors (RECIST) (Ollivier et al., 2001). Patients evaluated using RECIST can have a complete response (CR), a partial response (PR), stable disease (SD), or progressive disease (PD). An improved response can also be assessed by other criteria, for example, duration of response, reduction in tumor volume, minimum residual disease (MRD), and the like.

Patients to whom the methods and uses of the invention can be applied may be undergoing ICB therapy for any type of neoplastic disease or disorder wherein 4PD1^{hi} cells expand and contribute to immune suppression and immune refractoriness. In some embodiments, the 4PD1^{hi} cell frequency increases in a patient during the course of ICB therapy, relative to baseline. In this context, “baseline” is the 4PD1^{hi} cell frequency prior to treatment with the ICB therapy and/or BCL6 inhibitor, or prior to treatment with the ICB therapy and/or EZH2 inhibitor. In some embodiments, the 4PD1^{hi} cell frequency in a patient, either at baseline or during treatment, is significantly higher than the average 4PD1^{hi} cell frequency in healthy subjects. Examples of neoplastic diseases or disorders that can be treated by the methods of the invention include melanoma, skin carcinoma, NSCLC, kidney cancer, bladder cancer, head and neck cancers, lymphoma, breast cancer, ovarian cancer, prostate cancer, pancreatic cancer, colorectal cancer, gastric cancer, and esophageal cancer.

A “neoplastic cell” or “neoplasm” typically has undergone some form of mutation/transformation, resulting in abnormal growth as compared to normal cells or tissue of the same type. Neoplasms include morphological irregularities, as well as pathologic proliferation. Neoplastic cells can be benign or malignant. Malignant neoplasms, i.e., cancers, are distinguished from benign in that they demonstrate loss of differentiation and orientation of cells, and have the properties of invasion and metastasis.

A “solid tumor” is a mass of neoplastic cells.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already with the disorder. In certain embodiments, a subject is successfully “treated” for a disease or disorder according to the methods provided herein if the patient shows, e.g., total, partial, or transient alleviation or elimination of symptoms associated with the disease or disorder.

The terms “inhibit,” “block,” and “suppress” are used interchangeably and refer to any statistically significant decrease in biological activity, including full blocking of the activity. An “inhibitor” is an active agent that inhibits, blocks, or suppresses biological activity in vitro or in vivo. Inhibitors include but are not limited to small molecule compounds; nucleic acids, such as siRNA and shRNA; polypeptides, such as antibodies or antigen-binding fragments thereof, dominant-negative polypeptides, peptidomimetics, and inhibitory peptides; and oligonucleotide or peptide aptamers.

An “active agent” is an agent which itself has biological activity, or which is a precursor or prodrug that is converted in the body to an agent having biological activity.

An “effective amount” of an active agent or composition as disclosed herein is an amount sufficient to carry out a specifically stated purpose. An “effective amount” can be determined empirically and in a routine manner, in relation to the stated purpose, route of administration, and dosage form.

A “CTLA-4 inhibitor” is an active agent that antagonizes the activity of cytotoxic T lymphocyte-associated antigen 4 or reduces its production in a cell. Examples of CTLA-4 inhibitors include ipilimumab and tremelimumab. Derivatives of these compounds that act as CTLA-4 inhibitors are also suitable for use in the invention.

A “PD-1 inhibitor” is an active agent that antagonizes the activity of programmed cell death protein 1 or reduces its production in a cell. Examples of PD-1 inhibitors that are suitable for use in the present invention include nivolumab, pembrolizumab, pidilizumab, and REGN2810. PD-1 inhibitors also include active agents that inhibit the PD-1 ligand (PD-L1), including atezolizumab, avelumab, durvalumab, and BMS-936559. Derivatives of the foregoing compounds that act as PD-1 inhibitors are also suitable for use in the invention.

A “BCL6 inhibitor” is an active agent that antagonizes the activity of B-cell lymphoma 6 or reduces its production in a cell. A BCL6 inhibitor can target BCL6 or can target BCL6 binding partners/co-repressors. Examples of BCL6 inhibitors include, but are not limited to, 79.6, Apt48, BI-3812, BBD-BPI, L-BPI, RI-BPI, FX1, Resveratrol, Rifamycin SV, SMRT¹⁴¹⁴⁻¹⁴⁴¹, TMX-1120, TMX-2164, and derivatives thereof (Cerchietti et al., 2010; Cardenas et al., 2017 and references cited therein; Kerres et al., 2017; Teng et al., 2020).

An “EZH2 inhibitor” is an active agent that antagonizes the activity of EZH2 or reduces its production in a cell. An EZH2 inhibitor can target EZH2 or can target EZH2 binding partners/co-repressors. Examples of EZH2 inhibitors include, but are not limited to, EPZ-6438 (tazemetostat), GSK2816126, CPI-0209, SHR2554, and PF-06821497 (Kang et al., 2020). EZH2 inhibitors may also be dual EZH2/1 inhibitors such as CPI-1205 or DS-3201.

T_{FH} and 4PD1^{hi} cell frequencies are measured as a percentage of CD4⁺ T cells in a biological sample from a subject, in particular, in a peripheral blood sample or a tumor biopsy. Cell frequency can be measured or quantified by any method known in the art. Examples of suitable techniques include, but are not limited to, those that involve immunohistochemistry (IHC), flow cytometry, and/or transcriptome analysis, each of which technique can be used to detect, measure, and/or quantify cells having a given gene expression signature.

T_{FH} and/or 4PD1^{hi} cell function refers to the capacity of these cells to suppress T cell activation and proliferation. For example, even when BCL6 inhibition or EZH2 inhibition or genetic inactivation of CXCR5 does not lead to substantial reduction of T_{FH}/4PD1^{hi} cell frequency in the tumor microenvironment, it does inhibit the capacity of tumor-infiltrating 4PD1^{hi} cells to suppress the anti-tumor immune response. Accordingly, T_{FH} and/or 4PD1^{hi} cell function can be measured, for example, in T cell proliferation/suppression assays as described herein. In certain embodiments, T_{FH} and/or 4PD1^{hi} cells from clinical samples can be sorted by FACS and tested in ex vivo suppression assays to measure their capacity to suppress proliferation and activation of T cells in a co-culture system.

T_{FH} and/or 4PD1^{hi} cell frequency and/or function can be measured according to the methods of the invention at least about one, two, three, four, five, or six weeks after a dose of ICB therapy. In some cases, T_{FH} and/or 4PD1^{hi} cell frequency and/or function is measured before the first dose of the agent that reduces frequency or function of T_{FH} cells and/or 4PD1^{hi} cells to determine a patient's baseline T_{FH} and/or 4PD1^{hi} cell frequency and/or function. Because ICB therapy is typically cyclical (for example, one dose is administered every three weeks for a total of four doses), a baseline T_{FH} and/or 4PD1^{hi} cell frequency and/or function can be acquired before the first dose and/or before one or more subsequent doses of the agent.

As used herein, the term “gene expression signature” is used consistently with its conventional meaning in the art, and refers to an expression profile of a group of genes that is characteristic of a certain cell type, a certain cell population, a certain biological phenotype, or a certain medical condition. By way of example, when the term “gene expression signature” is used in relation to T_{FH} cells, it refers to an expression profile of a group of genes that is characteristic of T_{FH} cells. For example, T_{FH} cells are CXCR5-positive CD4-positive, PD-1-positive, Foxp3-negative, and BCL6-positive, i.e., T_{FH} cells can be characterized by the “gene expression signature” CXCR5'⁰ CD4+PD-1⁺Foxp3⁻BCL6+. Likewise, 4PD1l cells are CD4-positive, Foxp3-negative, and PD-1-positive, i.e., 4PD1^{hi} cells can be characterized by the “gene expression signature” CD4⁺Foxp3⁻PD-1+. Gene expression signatures can be determined using any suitable method known in the art for determining the expression of a gene, including, but not limited to, those that detect and/or measure gene expression at the mRNA level or the protein level, such as RT-PCR-based methods, immunohistochemistry (IHC)-based methods, flow cytometry-based methods, and the like.

The term “pharmaceutical composition” refers to a preparation that is in such form as to permit the biological activity of the active ingredient to be effective, and which contains no additional components that are unacceptably toxic to a subject to which the composition would be administered. Pharmaceutical compositions can be administered in any of numerous dosage forms, for example, tablet, capsule, liquid, solution, softgel, suspension, emulsion, syrup, elixir, tincture, film, powder, hydrogel, ointment, paste, cream, lotion, gel, mousse, foam, lacquer, spray, aerosol, inhaler, nebulizer, ophthalmic drops, patch, suppository, and/or enema. Pharmaceutical compositions typically comprise a pharmaceutically acceptable carrier, and can comprise one or more of a buffer (e.g., acetate, phosphate, or citrate buffer), a surfactant (e.g., polysorbate), a stabilizing agent (e.g., human albumin), a preservative (e.g., benzyl alcohol), a penetration enhancer, an absorption promoter to enhance bioavailability and/or other conventional solubilizing or dispersing agents. Choice of dosage form and excipients depends upon the active agent to be delivered and the disease or disorder to be treated or prevented, and is routine to one of ordinary skill in the art.

“Systemic administration” means that a pharmaceutical composition is administered such that the active agent enters the circulatory system, for example, via enteral, parenteral, inhalational, or transdermal routes. Enteral routes of administration involve the gastrointestinal tract and include, without limitation, oral, sublingual, buccal, and rectal delivery. Parenteral routes of administration involve routes other than the gastrointestinal tract and include, without limitation, intravenous, intramuscular, intraperitoneal, intrathecal, and subcutaneous. “Local administration” means that a pharmaceutical composition is administered directly to where its action is desired (e.g., at or near the site of the injury or symptoms). Local routes of administration include, without limitation, topical, inhalational, subcutaneous, ophthalmic, and otic. It is within the purview of one of ordinary skill in the art to formulate pharmaceutical compositions that are suitable for their intended route of administration.

In some embodiments, administration can comprise systemic administration, at any suitable dose and/or according to any suitable dosing regimen, as determined by one of skill in the art. The ICB therapy, for example, a CTLA-4 inhibitor and/or a PD-1 inhibitor, and the agent that reduces frequency or function of T_{FH} cells and/or 4PD1^{hi} cells can be administered according to any suitable dosing regimen, for example, where the daily dose of one or both agents is divided into two or more separate doses. It is within the skill of the ordinary artisan to determine a dosing schedule and duration for administration.

Initially, the agent that reduces frequency or function of T_{FH} cells and/or 4PD1^{hi} cells can be administered 1, 2, 3, 4, 5, 6, or 7 days prior to administration of the ICB therapy, preferably 1, 2, 3, or 4 days prior to administration of the ICB therapy, such as a CTLA-4 inhibitor and/or a PD-1 inhibitor. Alternatively, the agent can be initially administered on the same day as the ICB therapy.

The agent that reduces frequency or function of T_{FH} cells and/or 4PD1^{hi} cells is preferably administered throughout ICB therapy, i.e., administration of a course of treatment with an agent that reduces frequency or function of T_{FH} cells and/or 4PD1^{hi} cells is concurrent with administration of a course of ICB therapy. In one embodiment, the agent is administered once daily. In one embodiment, the agent is administered twice daily. In one embodiment, the agent is administered one, two, three, or four times per week. In one embodiment, the agent is administered every other day. In one embodiment, the agent is administered every three days.

Embodiments of the present disclosure can be further defined by reference to the following non-limiting examples. It will be apparent to those skilled in the art that many modifications, both to materials and methods, can be practiced without departing from the scope of the present disclosure.

### EXAMPLES

### Example 1. Targeting T_{F}H Cells to Improve Activity of ICB

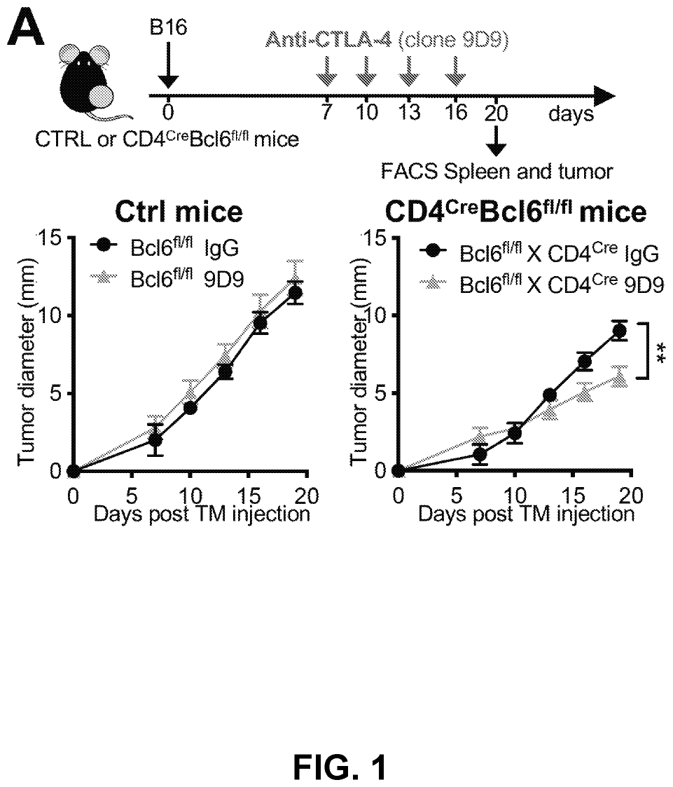

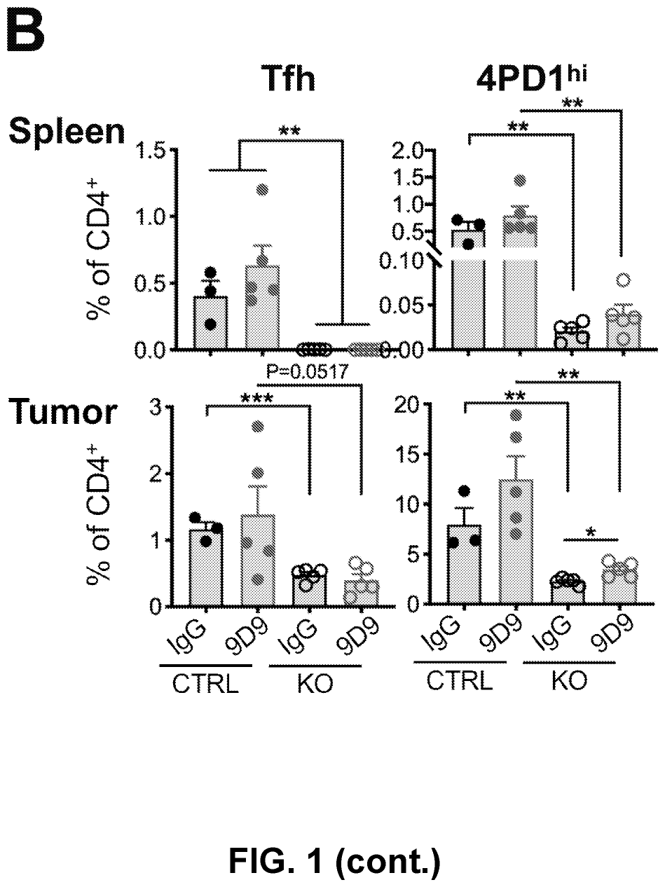

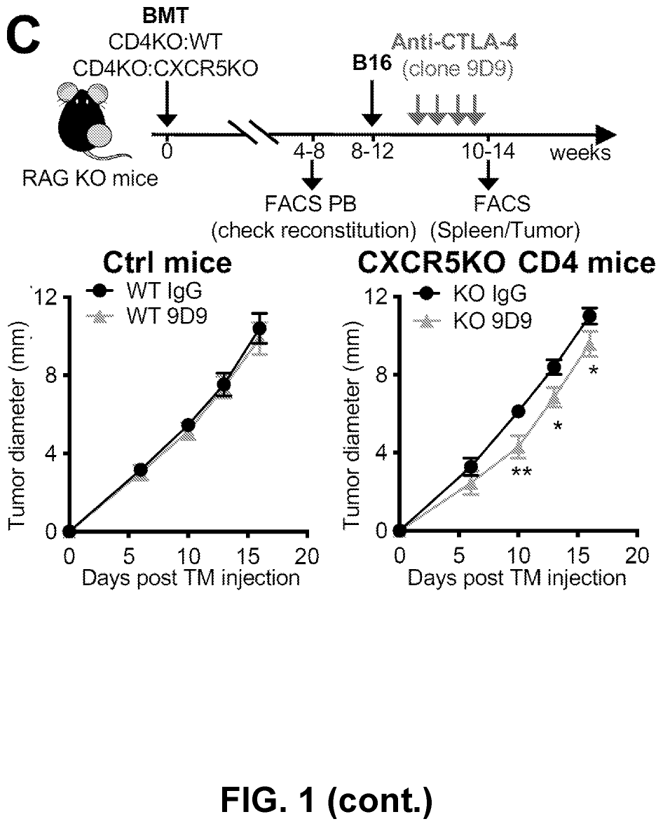

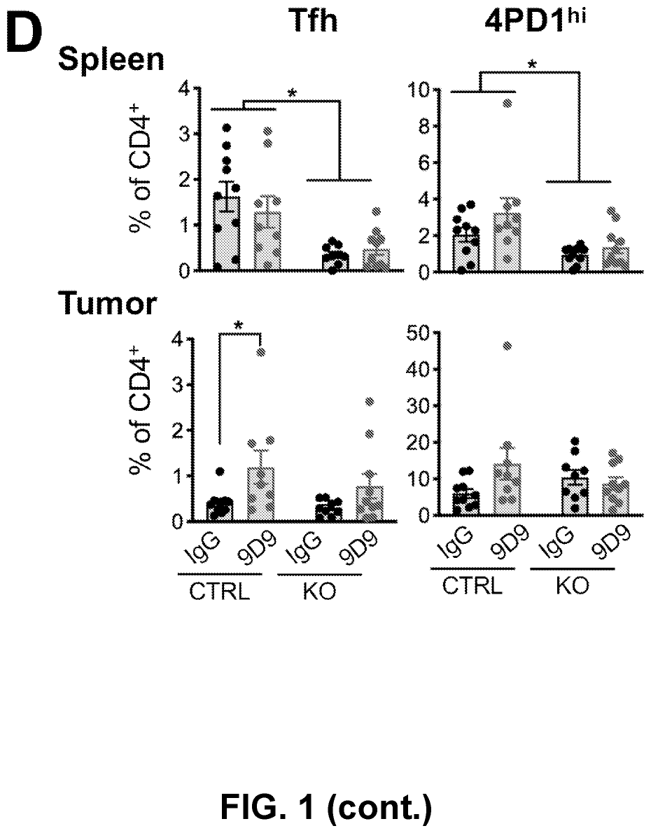

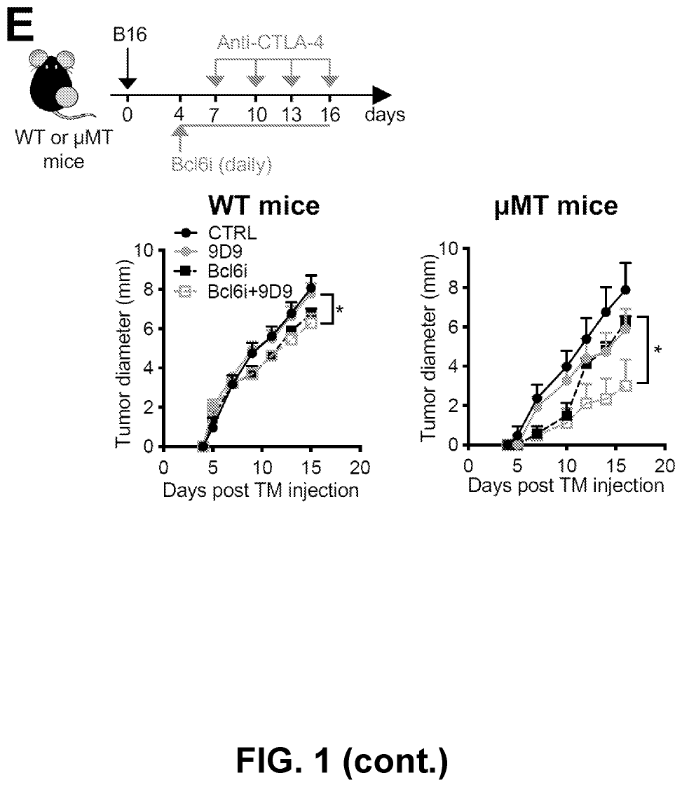

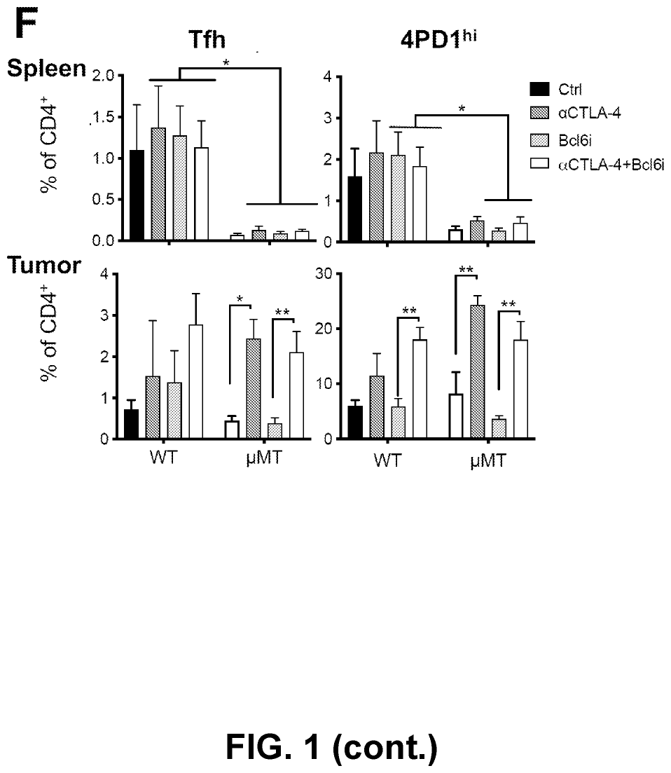

We have developed and employed the following genetic systems to eliminate T_{FH} cells, and to test whether immune checkpoint blockade can achieve better anti-tumor activity in the absence of T_{FH} cells: 1) a CD4 conditional Bcl6 knock out (KO) mouse model (CD4-Cre;Bcl6^{fl/fl}) (FIG. 1, Panels A, B) and 2) a CD4KO:CXCR5KO mixed bone marrow chimera system (FIG. 1, Panels C, D). Mice were implanted with the aggressive melanoma model B16F10 (B16) and treated with a suboptimal anti-CTLA-4 regimen starting 7 days after tumor implantation, which does not produce substantial tumor control in wild type mice (Zappasodi et al., 2018).

We determined that elimination of T_{FH} cells using either system potentiated the anti-tumor activity of this suboptimal anti-CTLA-4 regimen (FIG. 1, Panels A, C). As expected, the frequency of 4PD1^{hi} T_{FH}-like cells, as well as bonafide T_{FH} cells, was significantly reduced in the spleens of CD4-Cre;Bcl6^{fl/fl} mice and in CD4KO:CXCR5KO bone marrow transplant (BMT) recipients, independent of treatment (FIG. 1, Panels B, D).

Intriguingly, deletion of the T_{FH} lineage did not completely eliminate intratumor infiltration with PD-1-overexpressing CD4⁺Foxp3⁻ T cells (FIG. 1, Panels B, D). This was particularly evident in CD4KO:CXCR5 BMT recipient mice, where the frequency of intratumor 4PD1^{hi} cells was not substantially reduced compared to that in control mice, despite the fact that anti-CTLA-4 could not increase 4PD1^{hi} cells in these mice (FIG. 1, Panel D).

We next tested whether we could reproduce these results with pharmacologic inhibition of Bcl6 using a selective inhibitor (Bcl6i) (Cerchietti et al., 2010) in combination with anti-CTLA-4. In these experiments, we used both WT and B cell-deficient μMT mice (FIG. 1, Panels E, F) to compare the effects of Bcl6i in the presence or absence of B cells, which are the other major cell subset expressing Bcl6. The anti-tumor activity of Bcl6i+anti-CTLA-4 was more pronounced in the absence of B cells in μMT mice (FIG. 1, Panel E), where 4PD1^{hi} and bonafide T_{FH} cells were also significantly reduced in the spleens (FIG. 1, Panel F). However, once again in this model, we found that tumor-infiltrating 4PD1^{hi}, or T cells with a bonafide T_{FH} phenotype, were not substantially reduced, and anti-CTLA-4 was still able to upregulate their frequency, even during Bcl6 inhibition in B cell-deficient mice (FIG. 1, Panel F).

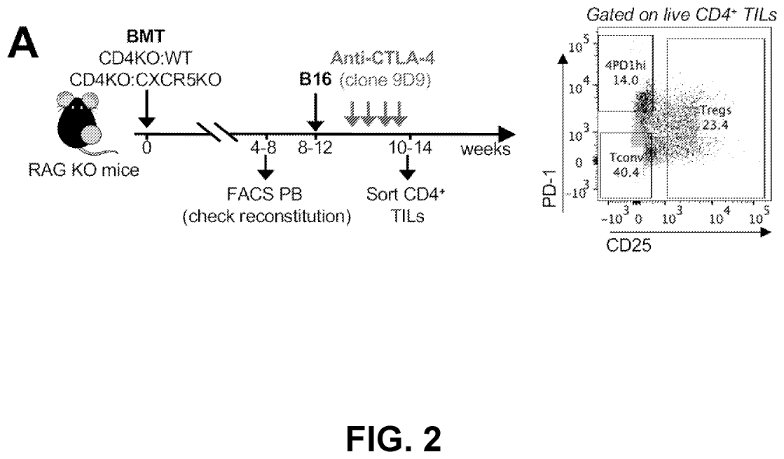

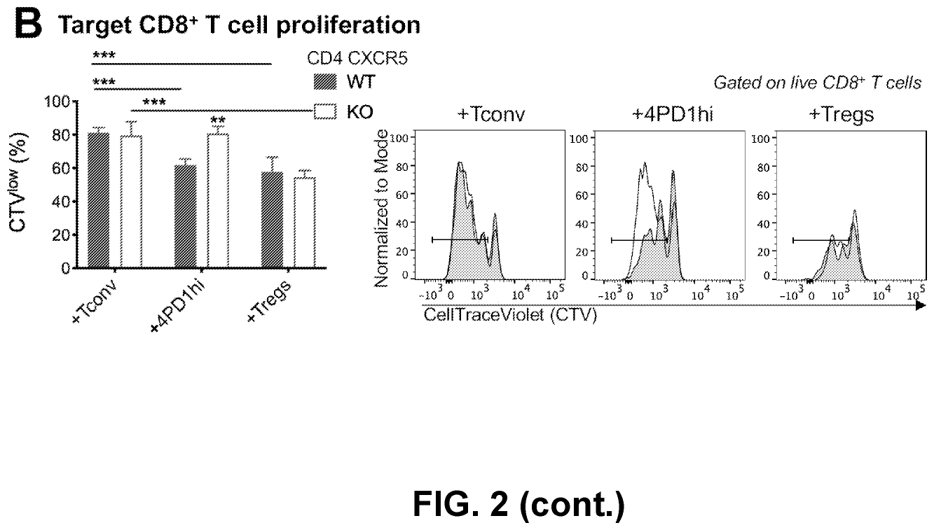

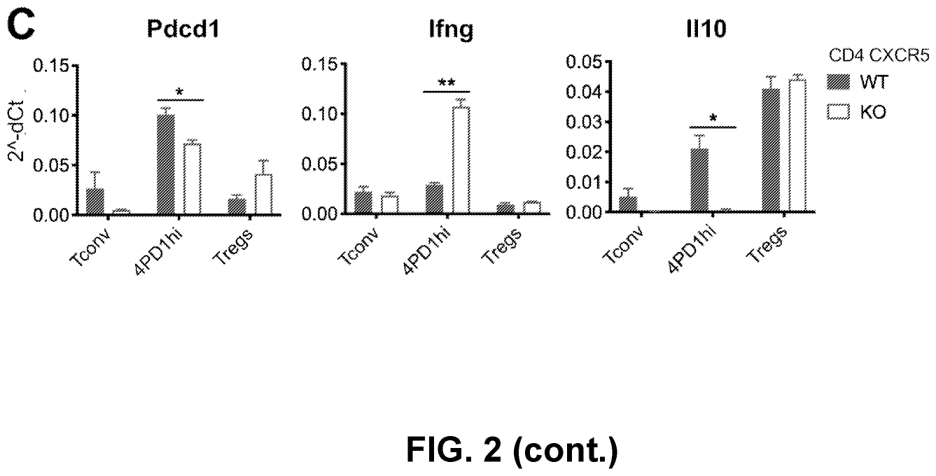

We thus reasoned that the 4PD1^{hi} cells found intratumorally after treatment with anti-CTLA-4 in T_{FH} deficient mice, or during Bcl6 inhibition in association with better tumor control, could be functionally different and have lost their suppression function. To test this hypothesis, we FACS-sorted 4PD1^{hi} from B16 tumors grown in CD4KO:CXCR5KO or CD4KO:WT BMT-recipient mice, treated with the same suboptimal anti-CTLA-4 regimen. We evaluated these cells in ex vivo suppression assays, in comparison with T_{regs} and conventional CD4⁺ T cells (Tconv) (FIG. 2, Panel A). In the absence of a Foxp3 reporter system in these mice, we used CD25 as a marker to distinguish T_{regs} from 4PD1^{hi} and Tconv, and to isolate these cell populations (FIG. 2, Panel A). In contrast to T_{regs} and similar to bonafide T_{FH} cells, 4PD1^{hi}, do not overexpress CD25 (Zappasodi et al., 2018). Therefore, CD25, in conjunction with PD-1, allows for reliable detection and separation of 4PD1^{hi} from T_{regs}.

We found that 4PD1^{hi} from tumors implanted in CD4KO:CXCR5KO BMT recipient mice were no longer suppressive, as opposed to the same cell subset isolated from tumors of control mice (FIG. 2, Panel B). In contrast, the function of Tconv and T_{regs} in these models did not differ (FIG. 2, Panel B). Importantly, we found that loss of suppression in tumor-infiltrating 4PD1^{hi} from CD4KO:CXCR5KO BMT-recipient mice was associated with reduced PD-1 and IL-10 expression levels, and upregulation of IFN-γ (FIG. 2, Panel C).

### Example 2. Functional Role of EZH2 in T_{FH}-Like Cells

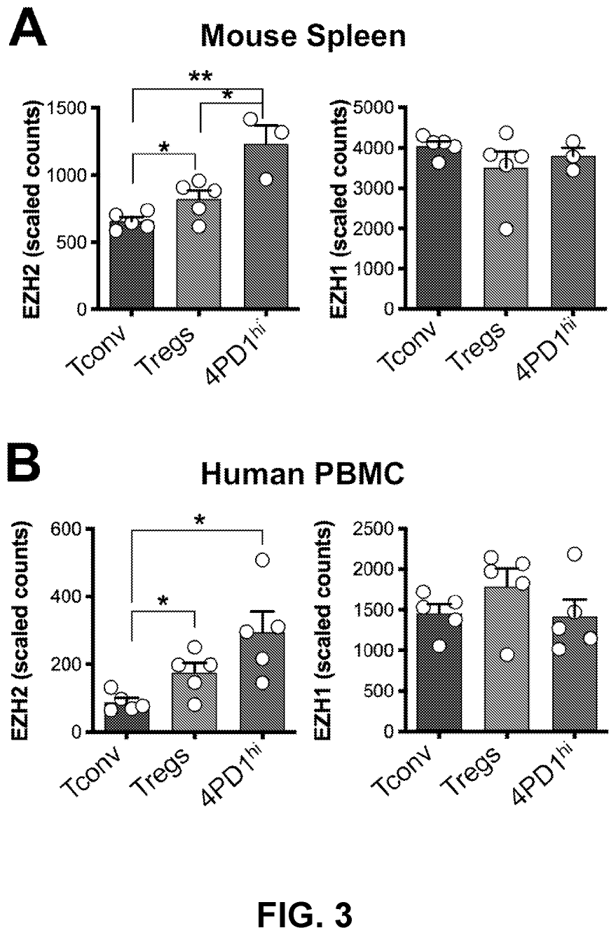

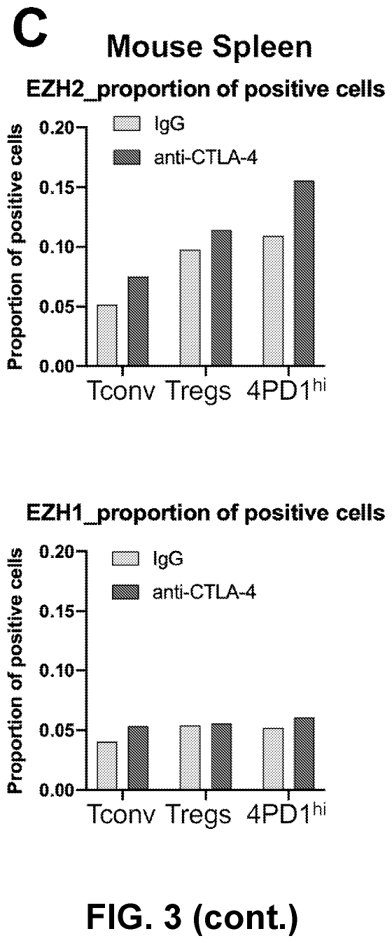

In consideration of the known effects of EZH2 in repressing transcription in cooperation with BCL6 in the germinal centers and its role in supporting immunosuppressive function of T_{regs}, we examined for EZH2 expression in 4PD1^{hi} cells in comparison with other CD4⁺ T-cell subsets. We found that, in naïve mouse spleen and in human healthy donor-derived peripheral monocyte blood cells, 4PD1^{hi} cells overexpress EZH2 (FIG. 3, Panels A and B). Notably, the expression levels of EZH2 in 4PD1^{hi} cells were at higher levels as compared to in conventional T cells (Tconv) and T_{regs} (FIG. 3, Panels A and B). Expression levels of EZH1 were similar across the T-cell subsets (FIG. 3, Panels A and B).

In addition, in mice treated with a CTLA-4 blocking antibody, EZH2 expression in 4PD1^{hi} cells is further upregulated as compared to mice treated with IgG control (FIG. 3, Panel C). In contrast, EZH1 was expressed at similar levels for each T-cell subset and treatment condition FIG. 3, Panel C)).

These results, in particular the preferential overexpression of EZH2 in 4PD1^{hi} cells and its further up-regulation in these cells after CTLA-4 blockade, suggest a functional role for EZH2 in 4PD1^{hi} cells.

### REFERENCES

Akiba H, et al. (2005). The role of ICOS in the CXCR5+follicular B helper T cell maintenance in vivo. Journal of Immunology 175, 2340-2348.
Avogadri F, et al. (2014). Combination of alphavirus replicon particle-based vaccination with immunomodulatory antibodies: therapeutic activity in the B16 melanoma mouse model and immune correlates. Cancer Immunology Research 2, 448-458.
Ballesteros-Tato A, et al. (2012). Interleukin-2 inhibits germinal center formation by limiting T follicular helper cell differentiation. Immunity 36, 847-856.
Barbie D A, et al. (2009). Systematic RNA interference reveals that oncogenic KRAS-driven cancers require TBK1. Nature 462, 108-112.
Baumjohann D, et al. (2013). Persistent antigen and germinal center B cells sustain T follicular helper cell responses and phenotype. Immunity 38, 596-605.
Béguelin W, et al. (2016). EZH2 and BCL6 cooperate to assemble CBX8-BCOR complex to repress bivalent promoters, mediate germinal center formation and lymphomagenesis. Cancer Cell 30, 197-213.
Budhu S, et al. (2010). CD8+ T cell concentration determines their efficiency in killing cognate antigen-expressing syngeneic mammalian cells in vitro and in mouse tissues. Journal of Experimental Medicine 207, 223-235.
Callahan M K, et al. (2013). Peripheral and tumor immune correlates in patients with advanced melanoma treated with combination nivolumab (anti-PD-1, BMS-936558, ONO-4538) and ipilimumab. Journal of Clinical Oncology 31, suppl; abstr 3003.
Cardenas M G, et al. (2016). Rationally designed BCL6 inhibitors target activated B cell diffuse large B cell lymphoma. Journal of Clinical Investigation 126, 3351-3362.
Cardenas, M G, et al. (2017). The Expanding Role of the BCL6 Oncoprotein as a Cancer Therapeutic Target. Clinical Cancer Research 23, 885-893.
Cerchietti L C, et al. (2010). A small-molecule inhibitor of BCL6 kills DLBCL cells in vitro and in vivo. Cancer Cell 17, 400-411.
Chen H, et al. (2009). Anti-CTLA-4 therapy results in higher CD4+ICOShi T cell frequency and IFN-gamma levels in both nonmalignant and malignant prostate tissues. Proceedings of the National Academy Science of the United States of America 106, 2729-2734.
Choi Y S, et al. (2015). LEF-1 and TCF-1 orchestrate T(FH) differentiation by regulating differentiation circuits upstream of the transcriptional repressor Bcl6. Nature Immunology 16, 980-990.
Crotty S. (2014). T follicular helper cell differentiation, function, and roles in disease. Immunity 41, 529-542.
Cubas R A, et al. (2013). Inadequate T follicular cell help impairs B cell immunity during HIV infection. Nature Medicine 19, 494-499.
Dong H, et al. (2002). Tumor-associated B7-H1 promotes T-cell apoptosis: a potential mechanism of immune evasion. Nature Medicine 8, 793-800.
duPage M, et al. (2015). The chromatin-modifying enzyme EZH2 is critical for the maintenance of regulatory T cell identity after activation. Immunity 42, 227-238.
Ellestad K K, et al. (2014). PD-1 is not required for natural or peripherally induced regulatory T cells: Severe autoimmunity despite normal production of regulatory T cells. European Journal of Immunology 44, 3560-3572.
Fife B T and Bluestone J A. (2008). Control of peripheral T-cell tolerance and autoimmunity via the CTLA-4 and PD-1 pathways. Immunological Reviews 224, 166-182.
Friedman C F, et al. (2016). Treatment of the Immune-Related Adverse Effects of Immune Checkpoint Inhibitors: A Review. JAMA Oncology 2, 1346-1353.
Gagliani N, et al. (2013). Coexpression of CD49b and LAG-3 identifies human and mouse T regulatory type 1 cells. Nature Medicine 19, 739-746.
Hale J S and Ahmed R. (2015). Memory T follicular helper CD4 T cells. Frontiers in Immunology 6, 16.
Hanzelmann S, et al. (2013). GSVA: gene set variation analysis for microarray and RNA-seq data. BMC Bioinformatics 14, 7.
He J, et al. (2013). Circulating precursor CCR7(lo)PD-1(hi) CXCR5(+) CD4(+) T cells indicate Tfh cell activity and promote antibody responses upon antigen reexposure. Immunity 39, 770-781.
He R, et al. (2016). Follicular CXCR5-expressing CD8+ T-cells curtail chronic viral infection. Nature 537, 412-428.
Hellmann M D, et al. (2016). Nivolumab plus ipilimumab as first-line treatment for advanced non-small-cell lung cancer (CheckMate 012): results of an open-label, phase 1, multicohort study. The Lancet Oncology 18, 31-41.
Hodi F S, et al. (2010). Improved survival with ipilimumab in patients with metastatic melanoma. New England Journal of Medicine 363, 711-723.
Holmgaard R B, et al. (2015). Tumor-Expressed IDO Recruits and Activates MDSCs in a Teg-Dependent Manner. Cell Reports 13, 412-424.
Hou T Z, et al. (2015). A transendocytosis model of CTLA-4 function predicts its suppressive behavior on regulatory T cells. Journal of Immunology 194, 2148-2159.
Im S J, et al. (2016). Defining CD8+ T cells that provide the proliferative burst after PD-1 therapy. Nature 537, 417-421.
Iwai Y, et al. (2002). Involvement of PD-L1 on tumor cells in the escape from host immune system and tumor immunotherapy by PD-L1 blockade. Proceedings of the National Academy Science of the United States of America 99, 12293-12297.
Johnston R J, et al. (2012). STAT5 is a potent negative regulator of TFH cell differentiation. Journal of Experimental Medicine 209, 243-250.
Kageyama R, et al. (2012). The receptor Ly108 functions as a SAP adaptor-dependent on-off switch for T cell help to B cells and NKT cell development. Immunity 36, 986-1002.
Kang N, et al. (2020). EZH2 inhibition: a promising strategy to prevent cancer immune editing. Epigenomics 12, 1457-1476.
Keir M E, et al. (2008). PD-1 and its ligands in tolerance and immunity. Annual Review of Immunology 26, 677-704.
Kenefeck R, et al. (2015). Follicular helper T cell signature in type 1 diabetes. Journal of Clinical Investigation 125, 292-303.
Kerres N, et al. (2017). Chemically Induced Degradation of the Oncogenic Transcription Factor BCL6. Cell Reports 20, 2860-2875.
Larkin J, et al. (2015). Combined Nivolumab and Ipilimumab or Monotherapy in Untreated Melanoma. New England Journal of Medicine 373, 23-34.
Leach D R, et al. (1996). Enhancement of antitumor immunity by CTLA-4 blockade. Science 271, 1734-1736.
Liu X, et al. (2012). Bcl6 expression specifies the T follicular helper cell program in vivo. Journal of Experimental Medicine 209, 1841-1852, S1841-1824.
Liu Y, et al. (2014). FoxA1 directs the lineage and immunosuppressive properties of a novel regulatory T cell population in EAE and MS. Nature Medicine 20, 272-282.
Lutsky J, et al. (2014). A phase 1 study of MEDI4736, and anti-PD-L1 antibody, in patients with advanced solid tumors. Journal of Clinical Oncology 32, suppl; abstr 3001.
Ma C S, et al. (2012). The origins, function, and regulation of T follicular helper cells. Journal of Experimental Medicine 209, 1241-1253.
Miyauchi K, et al. (2016). Protective neutralizing influenza antibody response in the absence of T follicular helper cells. Nature Immunology 17, 1447-1458.
Mylnarczyk C, et al. (2019). Germinal center-derived lymphomas: the darkest side of humoral immunity. Immunological Reviews 288, 214-239.
Murphy T L, et al. (2013). Specificity through cooperation: BATF-IRF interactions control immune-regulatory networks. Nature Reviews Immunology 13, 499-509.
Ng Tang D, et al. (2013). Increased frequency of ICOS+ CD4 T cells as a pharmacodynamic biomarker for anti-CTLA-4 therapy. Cancer Immunology Research 1, 229-234.
Ollivier L, et al. (2001). International criteria for measurement of tumor response. Cancer Imaging 2, 31-32.
Pentcheva-Hoang T, et al. (2004). B7-1 and B7-2 selectively recruit CTLA-4 and CD28 to the immunological synapse. Immunity 21, 401-413.
Pollock P M, et al. (2003). Melanoma mouse model implicates metabotropic glutamate signaling in melanocytic neoplasia. Nature Genetics 34, 108-112.
Postow M A, et al. (2015). Nivolumab and ipilimumab versus ipilimumab in untreated melanoma. New England Journal of Medicine 372, 2006-2017.
Rizvi N, et al. (2015). Safety and efficacy of first-line nivolumab (NIVO: anti-programmed death-1 [PD-1]) and ipilimumab in non-small cell lung cancer (NSCLC). Journal of Thoracic Oncology 10, suppl. 2; abstr 786.
Robert C, et al. (2015). Pembrolizumab versus Ipilimumab in Advanced Melanoma. New England Journal Medicine 372, 2521-2532.
Sage P T, et al. (2014a). Circulating T follicular regulatory and helper cells have memory-like properties. Journal of Clinical Investigation 124, 5191-5204.
Sage P T, et al. (2013). The receptor PD-1 controls follicular regulatory T cells in the lymph nodes and blood. Nature Immunology 14, 152-161.
Sage P T, et al. (2014b). The coinhibitory receptor CTLA-4 controls B cell responses by modulating T follicular helper, T follicular regulatory, and T regulatory cells. Immunity 41, 1026-1039.
Sahoo A, et al. (2015). Batf is important for IL-4 expression in T follicular helper cells. Nature Communications 6, 7997.
Seth S, et al. (2009). Abundance of follicular helper T cells in Peyer's patches is modulated by CD155. European Journal of Immunology 39, 3160-3170.

Strome S E, et al. (2003). B7-H1 blockade augments adoptive T-cell immunotherapy for squamous cell carcinoma. Cancer Research 63, 6501-6505.
Tangye S G, et al. (2013). The good, the bad and the ugly—TFH cells in human health and disease. Nature Reviews Immunology 13, 412-426.
Teng M, et al. (2020). Rationally Designed Covalent BCL6 Inhibitor That Targets a Tyrosine Residue in the Homodimer Interface. ACS Medicinal Chemistry Letters 11, 1269-1273.
Wang C J, et al. (2015). CTLA-4 controls follicular helper T-cell differentiation by regulating the strength of CD28 engagement. Proceedings of the National Academy Science of the United States of America 112, 524-529.
Weber J S, et al. (2015). Nivolumab versus chemotherapy in patients with advanced melanoma who progressed after anti-CTLA-4 treatment (CheckMate 037): a randomised, controlled, open-label, phase 3 trial. The Lancet Oncology 16, 375-384.
Wherry E J and Kurachi M. (2015). Molecular and cellular insights into T cell exhaustion. Nature Review Immunology 15, 486-499.
Wing J B, et al. (2014). Regulatory T cells control antigen-specific expansion of Tfh cell number and humoral immune responses via the coreceptor CTLA-4. Immunity 41, 1013-1025.
Wing K, et al. (2008). CTLA-4 control over Foxp3+regulatory T cell function. Science 322, 271-275.
Wolchok J D, et al. (2013). Nivolumab plus ipilimumab in advanced melanoma. New England Journal of Medicine 369, 122-133.
Yarilin D, et al. (2015). Machine-based method for multiplex in situ molecular characterization of tissues by immunofluorescence detection. Scientific Reports 5, 9534.
Zappasodi R and Merghoub T. (2015). Alphavirus-based vaccines in melanoma: rationale and potential improvements in immunotherapeutic combinations. Immunotherapy 7, 981-997.
Zappasodi R, et al. (2018). Non-conventional Inhibitory CD4⁺Foxp3⁻PD-1^{hi} Cells as a Biomarker of Immune Checkpoint Blockade Activity. Cancer Cell 33, 1017-1032.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance. The present invention is further described by the following claims.

1. A method of improving response of a patient to immune checkpoint blockade (ICB) therapy, the method comprising administering to the patient an agent that reduces frequency or function of T follicular helper (T_{FH}) cells and/or 4PD1^{hi} cells in the patient, wherein administration of the agent is commenced prior to or concurrently with the ICB therapy.

2. A pharmaceutical composition comprising an effective amount of an agent that reduces frequency or function of T follicular helper (T_{FH}) cells and/or 4PD1^{hi} cells in a patient for use in improving response of the patient to immune checkpoint blockade (ICB) therapy.

3. The method or composition of claim 1 or claim 2, wherein the agent comprises a B-cell lymphoma 6 (BCL6) inhibitor.

4. The method or composition of claim 3, wherein the BCL6 inhibitor is 79-6.

5. The method or composition of claim 1 or claim 2, wherein the agent comprises an enhancer of zeste homolog 2 (EZH2) inhibitor.

6. The method or composition of claim 5, wherein the EZH2 inhibitor is selected from EPZ-6438, GSK2816126, CPI-0209, SHR2554, and PF-06821497.

7. The method or composition of any one of claims 1 to 6, wherein the ICB therapy comprises a CTLA-4 inhibitor, a PD-1 inhibitor, or a combination thereof.

8. The method of claim 7, wherein the ICB therapy is a CTLA-4 inhibitor.

9. The method or composition of claim 8, wherein the CTLA-4 inhibitor is selected from the group consisting of ipilimumab and tremelimumab.

10. The method or composition of any one of claims 1 to 9, wherein the agent is administered to the patient 1-7 days prior to administration of the ICB therapy.

11. The method or composition of claim 10, wherein the agent is administered to the patient 1-4 days prior to administration of the ICB therapy.

12. The method or composition of any one of claims 1 to 9, wherein the agent is administered to the patient on the same day as the ICB therapy.

13. The method or composition of any one of claims 1 to 12, wherein a course of the agent is administered to the patient throughout a course of the ICB therapy.

14. A method of improving response of a patient to anti-CTLA-4 immune checkpoint blockade (ICB) therapy, the method comprising administering to the patient an effective amount of a B-cell lymphoma 6 (BCL6)inhibitor, wherein administration of the BCL6 inhibitor is commenced prior to or concurrently with administration of a CTLA-4 inhibitor.

15. A pharmaceutical composition comprising an effective amount of a B-cell lymphoma 6 (BCL6) inhibitor for use in improving response of a patient to anti-CTLA-4 immune checkpoint blockade (ICB) therapy.

16. The method or composition of claim 14 or claim 15, wherein the BCL6 inhibitor is 79-6.

17. The method or composition of any one of claims 14 to 16, wherein the CTLA-4 inhibitor is selected from the group consisting of ipilimumab and tremelimumab.

18. The method or composition of any one of claims 14 to 17, wherein the BCL6 inhibitor is administered to the patient 1-7 days prior to administration of the CTLA-4 inhibitor.

19. The method or composition of claim 18, wherein the BCL6 inhibitor is administered to the patient 1-4 days prior to administration of the CTLA-4 inhibitor.

20. The method or composition of any one of claims 14 to 17, wherein the BCL6 inhibitor is administered to the patient on the same day as the CTLA-4 inhibitor.

21. The method or composition of any one of claims 14 to 20, wherein a course of the BCL6 inhibitor is administered to the patient throughout a course of the ICB therapy.

22. A method of improving response of a patient to anti-CTLA-4 immune checkpoint blockade (ICB) therapy, the method comprising administering to the patient an effective amount of an enhancer of zeste homolog 2 (EZH2), wherein administration of the EZH2 inhibitor is commenced prior to or concurrently with administration of a CTLA-4 inhibitor.

23. A pharmaceutical composition comprising an effective amount of an enhancer of zeste homolog 2 (EZH2) inhibitor for use in improving response of a patient to anti-CTLA-4 immune checkpoint blockade (ICB) therapy.

24. The method or composition of claim 22 or claim 23, wherein the EZH2 inhibitor is selected from EPZ-6438, GSK2816126, CPI-0209, SHR2554, and PF-06821497.

25. The method or composition of any one of claims 22 to 24, wherein the CTLA-4 inhibitor is selected from the group consisting of ipilimumab and tremelimumab.

26. The method or composition of any one of claims 22 to 25, wherein the EZH2 inhibitor is administered to the patient 1-7 days prior to administration of the CTLA-4 inhibitor.

27. The method or composition of claim 26, wherein the EZH2 inhibitor is administered to the patient 1-4 days prior to administration of the CTLA-4 inhibitor.

28. The method or composition of any one of claims 22 to 25, wherein the EZH2 inhibitor is administered to the patient on the same day as the CTLA-4 inhibitor.

29. The method or composition of any one of claims 22 to 28, wherein a course of the EZH2 inhibitor is administered to the patient throughout a course of the ICB therapy.

30. The method or composition of any one of claims 1 to 29, wherein the frequency of T_{FH} cells and/or 4PD1^{hi} cells is measured using immunohistochemistry.

31. The method or composition of any one of claims 1 to 29, wherein the frequency of T_{FH} cells and/or 4PD1^{hi} cells is measured using flow cytometry.

32. The method or composition of claim 31, wherein the flow cytometry is fluorescence-activated cell sorting (FACS).

33. The method or composition of any one of claims 1 to 29, wherein the frequency of T_{FH} cells and/or 4PD1^{hi} cells is measured using gene expression signature.

34. The method or composition of any one of claims 1 to 33, wherein the frequency or function of T_{FH} cells and/or 4PD1^{hi} cells is measured from a peripheral blood sample from the patient.

35. The method or composition of any one of claims 1 to 33, wherein the frequency or function of T_{FH} cells and/or 4PD1^{hi} cells is measured from a tumor biopsy sample from the patient.

36. The method or composition of any one of claims 1 to 35, wherein the patient has cancer.

37. The method or composition of claim 36, wherein the cancer is melanoma.

38. The method or composition of claim 36, wherein the cancer is non-small cell lung cancer.
